(19) United States
(12) Patent Application Publication
(10) Pub. No.: US 2004/0147647 A1
(43) Pub. Date: Jul. 29, 2004

(54) Polyorganosiloxane micro-emulsion composition and raw material for cosmetics

(76) Inventor: Tadashi Hamachi, Prefecture (JP)

Correspondence Address: Robert L McKellar, PoseyvilleProfessional Complex, 784 South Poseyville Road, Midland, MI, 48640, US

(21) Appl. No.: 10/477,637

(22) PCT Filed: May 20, 2002
(86) PCT No.: PCT/JP02/04871

(51) Int. Cl. A61K 7/06; A61K 7/11

(52) U.S. Cl. 524/157; 424/70.12

(57) ABSTRACT

A polyorganosiloxane micro-emulsion composition comprising (A) a polyorganosiloxane, (B) an N-acylalkyltaurine and/or an N-acylalkyltaurine salt, and (C) water, the emulsion having an average particle size less than 0.15 μm; and a raw material for cosmetics comprising the aforesaid polyorganosiloxane micro-emulsion composition.

A polyorganosiloxane micro-emulsion composition comprising (A) a polyorganosiloxane, (B) an N-acylalkyltaurine and/or an N-acylalkyltaurine salt, (C) water, and (D) a nonionic surface active agent, the emulsion having an average particle size less than 0.15 μm; and a raw material for cosmetics comprising the aforesaid polyorganosiloxane micro-emulsion composition.

The present invention relates to a polyorganosiloxane micro-emulsion composition and a raw material for cosmetics comprising the polyorganosiloxane micro-emulsion composition. More specifically, it relates to a polyorganosiloxane micro-emulsion composition which is colorless and transparent and possesses cosmetic functionality offering superior moisturized feel and slip, and to a raw material for cosmetics of superior color tone stability and stability in mixtures with other raw materials.

### BACKGROUND ART

Polydiorganosiloxane emulsion compositions bearing various functional groups are widely used as a raw material for various cosmetics, such as shampoos, rinse-in shampoos, hair mousse, hair mist, skin creams, skin lotions, and hand creams. In particular, polyorganosiloxane micro-emulsion compositions with an average particle size of 0.15 μm or less are used in many cosmetics because of their excellent stability in mixtures and transparent appearance. For example, Japanese Patent Publication (Kokoku) No. Hei 04(1992)-062288 offers a cosmetic of superior storage stability and stability in mixtures in which a polydimethylsiloxane micro-emulsion is used as the main ingredient. In the past aliphatic-substituted benzenesulfonic acid represented by acids such as dodecylbenzenesulfonic acid have been used as surface active agents in such polydiorganosiloxane micro-emulsions. However, because such surface active agents exhibit strong detergent performance, compounding micro-emulsions containing them with cosmetics brings about a considerable deterioration in the skin feel due to irritation of the skin or scalp. In addition such micro-emulsions are typically light yellow to yellow in color, with their color tone deepening over time.

On the other hand a polyorganosiloxane micro-emulsion composition utilizing a mixture of tetradecenesulfonic acid, i.e. an unsaturated aliphatic sulfonic acid, and hydroxytetradecanesulfonic acid, i.e. a hydroxylated aliphatic sulfonic acid, has been offered in Japanese Patent Application Publication (Kokai) No. Hei 10(1998)-265577. However this micro-emulsion composition is subject to yellowing due to oxidation by emulsifying agents during production and during compounding with cosmetics, and as a result brings about a noticeable deterioration in the external appearance of the cosmetics.

It is an object of this invention to provide a polyorganosiloxane micro-emulsion composition possessing superior cosmetic functionality in terms of being colorless and transparent and giving a superior moisturized feel and slip, and to a raw material for cosmetics of superior color tone stability and stability in mixtures with other raw materials.

### DISCLOSURE OF INVENTION

The present invention relates to a polyorganosiloxane micro-emulsion composition comprising (A) a polyorganosiloxane, (B) an N-acylalkyltaurine and/or an N-acylalkyltaurine salt, and (C) water, the emulsion having an average particle size less than 0.15 μm; and to a raw material for cosmetics comprising the aforesaid polyorganosiloxane micro-emulsion composition.

Another present invention relates to a polyorganosiloxane micro-emulsion composition comprising (A) a polyorganosiloxane, (B) an N-acylalkyltaurine and/or an N-acylalkyltaurine salt, (C) water, and (D) a nonionic surface active agent, the emulsion having an average particle size less than 0.15 μm; and to a raw material for cosmetics comprising the aforesaid polyorganosiloxane micro-emulsion composition.

First of all, a detailed explanation is provided regarding the polyorganosiloxane micro-emulsion composition of the present invention.

Polyorganosiloxane (A), which is the main ingredient of the emulsion of the present invention, is a linear, partially branched linear, or branched polyorganosiloxane represented by the average unit formula (1): R¹ₙSiO_{(4-n/2)}-(I),

where R¹ are identical or different substituted or unsubstituted monovalent hydrocarbon groups specifically exemplified by methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, and other saturated aliphatic hydrocarbon groups; vinyl, allyl, hexenyl, and other unsaturated aliphatic hydrocarbon groups; cyclopentyl, cyclohexyl, and other saturated alicyclic hydrocarbon groups; phenyl, tolyl, naphthyl, and other aromatic hydrocarbon groups, as well as groups obtained by substituting halogen atoms or organic groups including epoxy, carboxyl, amino, methacrylic, and mercapto groups for some of the hydrogen atoms bonded to the carbon atoms of these groups. In addition, although some of R¹ may be hydroxyl and alkoxy groups, preferably, not less than 70%, and, even more preferably, not less than 80% of R¹ are methyl groups. This is due to the fact that in this range the surface tension of the polyorganosiloxane is low, which makes it possible to obtain micro-emulsion compositions of superior glossiness and excellent spreadability. Subscript n is a number greater than 0 and less than 4, but to obtain a high molecular weight polyorganosiloxane micro-emulsion composition with a low surface tension and superior spreadability, n should preferably be between 1 and 2.5, and even more preferably between 1.8 and 2.2. The number average molecular weight of component (A) at 25° C. is preferably in the range of 1,000 to 1,000,000, and even more preferably in the range of 5,000 to 1,000,000.

The N-acylalkyltaurine and/or salt thereof (B), which is used to emulsify component (A) in water, is represented by compounds described by the general formula

(V): R⁵CONR⁶CH₂CH₂SO₃M (V),

where R⁵ and R⁶ are identical or different substituted or unsubstituted monovalent hydrocarbon groups specifically exemplified by methyl, ethyl, propyl, butyl, pentyl, hexyl, heptyl, octyl, nonyl, decyl, dodecyl, myristyl, palmityl, stearyl, and other saturated aliphatic hydrocarbon groups; vinyl, allyl, hexenyl, oleyl, and other unsaturated aliphatic hydrocarbon groups; cyclopentyl, cyclohexyl, and other saturated alicyclic hydrocarbon groups; and by phenyl, tolyl, naphthyl, and other aromatic hydrocarbon groups. The number of carbon atoms in R⁵ is preferably 1 to 30. In addition, the number of carbon atoms in R⁶ is preferably 1 to 6, with the groups being mostly methyl. Substituent M is exemplified by hydrogen atoms, alkali metal atoms such as sodium and potassium; and ammonium and triethanolammonium. Component (B) is exemplified by N-lauroyl methyltaurine sodium salt, N-coconut oil fatty acid methyltaurine potassium salt, N-coconut oil fatty acid methyltaurine sodium salt, N-myristoyl methyltaurine sodium salt, N-palmitoyl methyltaurine sodium salt, N-stearoyl methyltaurine sodium salt, N-stearoyl methyltaurine potassium salt, N-oleoyl methyltaurine sodium salt, N-cetyloyl methyltaurine potassium salt, and their non-neutralized forms. One of the above compounds can be used singly or several compounds can be used in combination. In addition, when salts of N-acylalkyltaurine are used, sufficient catalytic activity for emulsion polymerization is not likely, and it is preferable to add an acid. The acid is a component that contributes to the polymerization of the organosiloxane by converting at least one of the N-acylalkyltaurine salts to an acidic type (sulfone groups) and is specifically exemplified by hydrochloric acid, sulfuric acid, phosphoric acid, and acetic acid. Among these acids, it is preferable to use sulfuric acid or hydrochloric acid, which exhibits a high degree of dissociation and a higher catalytic activity and result in a shorter emulsion polymerization time, as compared with acetic acid and phosphoric acid which exhibit a low degree of dissociation. The amount, in which component (B) is used is preferably 5 to 300 parts by weight, and even more preferably 5 to 200 parts by weight per 100 parts by weight of component (A). This is due to the fact that when it is less than 5 parts by weight, the average particle size of the emulsion composition of the present invention may become greater than 0.15 μm, and when it exceeds 300 parts by weight, the viscosity of the emulsion may become too high, resulting in a deterioration in its flowability and operating characteristics.

Component (C), water, serves as a medium for the emulsification of component (A) using component (B). The amount, in which water is added should be sufficient to turn the present composition into an O/W (oil in water) type micro-emulsion, which is preferably 10 to 1000 parts by weight, and even more preferably 50 to 1000 parts by weight per 100 parts by weight of component (A).

The present emulsion composition consists of the above-described component (A) to component (C). However, it is preferable to further add a nonionic surface active agent (D) as a component serving as an aid in the emulsification of component (A). Ethylene glycol fatty acid esters, poly(ethylene glycol) fatty acid esters, propylene glycol fatty acid esters, poly(propylene glycol) fatty acid esters, glycol fatty acid esters, trimethylolpropane fatty acid esters, pentaerythritol fatty acid esters, glucoside derivatives, glycerin alkyl ether fatty acid esters, trimethylolpropane oxyethylene alkyl ethers, fatty acid amides, alkylolamides, alkylamine oxides, lanolin and its derivatives, castor oil derivatives, hardened castor oil derivatives, sterols and its derivatives, polyoxyethylene alkyl ethers, polyoxyethylene alkyl allyl ethers, polyoxyethylene alkylamine, polyoxyethylene fatty acid amides, polyoxyethylene alkylolamides, polyoxyethylene diethanolamine fatty acid esters, polyoxyethylene trimethylolpropane fatty acid esters, polyoxyethylene alkyl ether fatty acid esters, polyoxyethylene polyoxypropylene glycols, polyoxyethylene polyoxypropylene alkyl ethers, polyoxyethylene polyoxypropylene polyhydric alcohol ethers, glycerin fatty acid esters, polyglycerin fatty acid esters, polyoxyethylene glycerin fatty acid esters, sorbitan fatty acid esters, polyoxyethylene sorbitan fatty acid esters, and sucrose fatty acid esters are suggested as specific examples. Among these, polyoxyethylene alkyl ethers are preferable. Such nonionic surface active agents can be used singly or as a combination of several agents. The amount, in which component (D) is added is preferably 5 to 200 parts by weight, and even more preferably 5 to 100 parts by weight per 100 parts by weight of component (A). This is due to the fact that when component (D) is less than 5 parts by weight, the average particle size of the present emulsion composition may become greater than 0.15 μm, and when it exceeds 200 parts by weight the viscosity of the emulsion may become too high resulting in a deterioration in operating properties.

The present emulsion composition as described above can be produced, for example, by subjecting a polyorganosiloxane of a lower molecular weight than component to emulsion polymerization in water in the presence of (B) an N-acylalkyltaurine and/or its salt, or subjecting a polyorganosiloxane of a lower molecular weight than component (A) to emulsion polymerization in water in the presence of (B) an N-acylalkyltaurine and/or its salt and (D) a nonionic surface active agent. Linear, branched, or cyclic polyorganosiloxanes are suggested as the polyorganosiloxanes of a lower molecular weight. The same substituted or unsubstituted monovalent hydrocarbon groups as the above-described R¹ are suggested as the organic groups bonded to silicon atoms of the lower molecular weight polyorganosiloxane. In addition, the polyorganosiloxane may contain silicon-bonded hydroxyl groups, alkoxy groups, or hydrogen atoms.

The linear or branched low molecular weight polyorganosiloxanes are exemplified by polyorganosiloxanes represented by the following general formula (II) and formula (III).

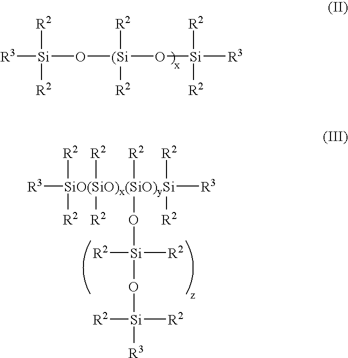

In the formulas, R² and R³ stand for identical or different substituted or unsubstituted monovalent hydrocarbon groups specifically exemplified by the same groups as the above-described R¹. In addition, some of R² and R³ may be hydroxyl groups, alkoxy groups, or hydrogen atoms. The subscripts x and z are integers of 0 to 100, with integers of 0 to 50 being preferable. The subscript y is an integer of 1 to 100, with an integer of 1 to 50 being preferable. Specific examples of such polyorganosiloxanes include α, ωdihydroxypolydimethylsiloxane, α,ω-dimethoxypolydimethylsiloxane, tetramethyl-1,3-dihydroxydisiloxane, octamethyl-1,7-dihydroxytetrasiloxane, hexamethyl-1,5-diethoxytrisiloxane, hexamethyldisiloxane, and octamethyltrisiloxane.

In addition, polyorganosiloxanes represented by the following general formula (IV) are suggested as cyclic low molecular weight polyorganosiloxanes.

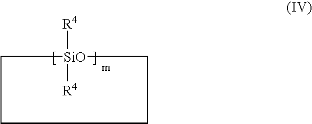

In the formula, R⁴ represents identical or different substituted or unsubstituted monovalent hydrocarbon groups specifically exemplified by the same groups as the above-described R¹. In addition, some of R⁴ may be hydroxyl groups or hydrogen atoms. The subscript m is an integer of 3 to 8. Specific examples of such cyclic polyorganosiloxanes include hexamethylcyclotrisiloxane, octamethylcyclotetrasiloxane, decamethylcyclopentasiloxane, dodecamethylcyclohexasiloxane, 1,1-diethylhexamethylcyclotetrasiloxane, phenylheptamethylcyclotetrasiloxane, 1,1-diphenylhexamethylcyclotetrasiloxane, 1,3,5,7-tetravinyltetramethylcyclotetrasiloxane, 1,3,5,7-tetramethylcyclotetrasiloxane, 1,3,5,7-tetracyclohexyltetramethylcyclotetrasiloxane, tris(3,3,3-trifluoropropyl)trimethylcyclotrisiloxane, 1,3,5,7-tetra(3-aminopropyl)tetramethylcyclotetrasiloxane, 1,3,5,7-tetra(N-(2-aminoethyl)-3-aminopropyl)tetramethylcyclotetrasiloxane, 1,3,5,7-tetra(3-mercaptopropyl)tetramethylcyclotetrasiloxane, 1,3,5,7-tetra(3-glycidoxypropyl)tetramethylcyclotetrasiloxane, 1,3,5,7-tetra(3-methacryloxypropyl)tetramethylcyclotetrasiloxane, 1,3,5,7-tetra(3-amidopropyl)tetramethylcyclotetrasiloxane, 1,3,5,7-tetra(3-acryloxypropyl)tetramethylcyclotetrasiloxane, 1,3,5,7-tetra(3-carboxypropyl)tetramethylcyclotetrasiloxane, 1,3,5,7-tetra(3-vinyloxypropyl)tetramethylcyclotetrasiloxane, 1,3,5,7-tetra(p-vinylphenyl)tetramethylcyclotetrasiloxane, 1,3,5,7-tetra {3-(p-vinylphenyl)propyl}tetramethylcyclotetrasiloxane, 1,3,5,7-tetra{3-(p-isopropenylbenzoylamino)propyl} tetramethylcyclotetrasiloxane, 1,3,5,7-tetra(N-methacryloyl-N-methyl-3-aminopropyl)tetramethylcyclotetrasiloxane, 1,3,5,7-tetra(N-lauroyl-N-methyl-3-aminopropyl)tetramethylcyclotetrasiloxane, 1,3,5,7-tetra(N-acryloyl-N-methyl -3-aminopropyl)tetramethylcyclotetrasiloxane, 1,3,5,7-tetra(N,N-bis(methacryloyl)-3-aminopropyl)tetramethylcyclotetrasiloxane, and 1,3,5,7-tetra(N,N-bis(lauroyl)-3-aminopropyl)tetramethylcyclotetrasiloxane.

In the present invention such polyorganosiloxanes of a lower molecular weight than component (A) can be used singly or as a combination of several polyorganosiloxanes. For example, it is possible to use cyclic polyorganosiloxanes as the main ingredient with linear polyorganosiloxanes mixed therewith. In such a case, there are no particular limitations concerning the proportion, in which they are to be combined. However, a proportion of cyclic to linear polyorganosiloxanes of 50 to 99.999 wt %:50 to 0.001 wt % is preferable, and a proportion of 70 to 99.999 wt %:30 to 0.001 wt % is even more preferable. The use of cyclic polyorganosiloxanes along with linear polyorganosiloxanes makes it possible to easily regulate the degree of polymerization of the polyorganosiloxane during emulsion polymerization.

Furthermore, during the emulsion polymerization, in addition to the above-described low molecular weight polyorganosiloxanes, hydrolyzable organosilanes bearing organic functional groups can be added. By doing so, it is possible to introduce organic functional groups into the polyorganosiloxane during the emulsion polymerization. Such hydrolyzable organosilanes are exemplified by 3-aminopropyldimethoxysilane, 3-aminopropyltrimethoxysilane, N-(2-aminoethyl)-3-aminopropyltrimethoxysilane, N-(2-aminoethyl)-3-aminopropyldimethoxysilane, 3-chloropropyltrimethoxysilane, 3-chloropropyldimethoxysilane, 3-methacryloxypropyltrimethoxysilane, 3-acryloxypropyldimethoxysilane, 3-glycidoxypropyltrimethoxysilane, 3-glycidoxypropylmethyldimethoxysilane, 3-mercaptopropylmethyldimethoxysilane, 3-carboxypropylmethyldimethoxysilane, p-vinylphenyltrimethoxysilane, 2-(vinylphenyl)ethyltrimethoxysilane, 3-(p-isopropenylbenzoylamino)propyltrimethoxysilane, N-methacryloyl-N-methyl-3-aminopropyltrimethoxysilane, N-lauroyl-N-methyl-3-aminopropylmethyldimethoxysilane, N-acryloyl-N-methyl-3-aminopropyltrimethoxysilane, N-lauroyl-N-methyl-3-aminopropyltrimethoxysilane, N,N-bis(methacryloyl)-3-aminopropylmethyldimethoxysilane, N,N-bis(lauroyl)-3-aminopropyltrimethoxysilane, 3-aminopropyldiethoxysilane, 3-aminopropyltriethoxysilane, N-(2-aminoethyl)-3-aminopropyltriethoxysilane, N-(2-aminoethyl)-3-aminopropyldiethoxysilane, 3-chloropropyltriethoxysilane, 3-chloropropyldiethoxysilane, 3-methacryloxypropyltriethoxysilane, 3-acryloxypropyldiethoxysilane, 3-glycidoxypropyltriethoxysilane, 3-glycidoxypropylmethyldiethoxysilane, 3-mercaptopropylmethyldiethoxysilane, 3-carboxypropylmethyldiethoxysilane, p-vinylphenyltriethoxysilane, 2-(vinylphenyl)ethyltriethoxysilane, 3-(p-isopropenylbenzoylamino)propyltriethoxysi lane, N-methacryloyl-N-methyl-3-aminopropyltriethoxysilane, N-lauroyl-N-methyl-3-aminopropylmethyldiethoxysilane, N-acryloyl-N-methyl-3-aminopropyltriethoxysilane, N-lauroyl-N-methyl-3-aminopropyltriethoxysilane, N,N-bis(methacryloyl)-3-aminopropylmethyldiethoxysilane, and N,N-bis(lauroyl)-3-aminopropyltriethoxysilane. The compounds above can be used singly or as a mixture of two or more compounds.

For conducting the emulsion polymerization one can use ordinary methods such as, for example, a method in which a surface active agent, a polymerization catalyst, and water are mixed and a coarse emulsion consisting of a cyclic polyorganosiloxane, a surface active agent, and water is added thereto (see Japanese Patent Application Publication (Kokai) No. Sho 62(1987)-141029); a method in which a mixture consisting of a surface active agent, a polymerization catalyst, and water is subjected to agitation and a cyclic polyorganosiloxane is added thereto (Japanese Patent Application Publication (Kokai) No. Hei 10(1998)-265577); and a method in which a polyorganosiloxane is dispersed in water in the presence of a surface active agent and a polymerization catalyst and ultrasonic wave energy is applied during the process of emulsion polymerization (Japanese Patent Application Publication (Kokai) No. Hei 04(1992)-103631). When a surface active agent exhibiting a polymerizing action on the polyorganosiloxane is used in these methods the use of the polymerization catalyst is unnecessary.

The present polyorganosiloxane micro-emulsion composition comprises the above-described component (A) to component (C) or component (A) to component (D). However, in order to further reduce the average particle size of the emulsion and further improve its stability in mixtures with other cosmetic materials, it is possible to add and combine with the composition other components known as additives for polyorganosiloxane micro-emulsion compositions so long as this addition does not impair the objective of the present invention. Such additives are exemplified by anionic surface active agents other than those described in component (B), pH-regulating agents, antiseptics, mildew-proofing agents, and rust preventives. These components can be used singly or as a combination of several compounds.

Specific examples of the anionic surface active agents include N-acyl-L-glutamic acid diethanolamine, N-acyl-L-glutamic acid triethanolamine, sodium N-acyl-L-glutamate, sodium alkanesulfonate, ammonium alkyl (12,14,16) sulfate, alkyl (11,13,15) sulfuric acid triethanolamine (1), alkyl (11,13,15) sulfuric acid triethanolamine (2), alkyl (12 to 14) sulfuric acid triethanolamine, liquid alkylsulfuric acid triethanolamine, sodium alkyl (12, 13) sulfate, liquid sodium alkylsulfate, sodium isoethionate, sodium lacto-isostearate, disodium undecylenoylamido ethyl sulfosuccinate, triethanolamine sulfooleate, sodium sulfooleate, disodium oleamide sulfosuccinate, potassium oleate, sodium oleate, morpholine oleate, oleoyl sarcosine, oleoyl methyltaurine sodium salt, potassium-containing soap base, liquid base for potassium soap, potassium soap, carboxylated polyoxyethylene tridodecyl ether, sodium salt (3 E.O.) of carboxylated polyoxyethylene tridodecyl ether, triethanolamine N-hydrogenated tallow fatty-acyl-L-glutamate, sodium N-hydrogenated tallow fatty-acyl-L-glutamate, sodium hydrogenated coconut fatty acid glyceryl sulfate, sodium diundecylenoylamido ethyl sulfosuccinate, sodium stearyl sulfate, potassium stearate, triethanolamine stearate, sodium stearate, sodium N-stearoyl-L-glutamate, disodium stearoyl-L-glutamate, stearoyl methyltaurine sodium salt, sodium dioctyl sulfosuccinate, liquid sodium dioctyl sulfosuccinate, liquid disodium polyoxyethylene monooleylamido sulfosuccinate (2 E.O.), disodium polyoxyethylene lauroyl ethanolamide sulfosuccinate (5 E.O.), disodium lauryl sulfosuccinate, diethanolamide cetyl sulfate, sodium cetyl sulfate, soap base, sodium cetostearyl sulfate, triethanolamine tridecyl sulfate, potassium palmitate, sodium palmitate, palmitoyl methyltaurine sodium salt, liquid castor oil fatty acid sodium salt (30%), ammonium polyoxyethylene alkyl ether sulfate (3 E.O.), liquid diethanolamine polyoxyethylene alkyl (12, 13) ether sulfate, liquid triethanolamine polyoxyethylene alkyl ether sulfate (3 E.O.), triethanolamine polyoxyethylene alkyl (11, 13, 15) ether sulfate (1 E.O.), triethanolamine polyoxyethylene alkyl (12, 13) ether sulfate (3 E.O.), liquid sodium polyoxyethylene alkyl ether sulfate (3 E.O.), sodium polyoxyethylene alkyl (11, 13, 15) ether sulfate (1 E.O.), sodium polyoxyethylene alkyl (111 to 15) ether sulfate (3 E.O.), sodium polyoxyethylene alkyl (12, 13) ether sulfate (3 E.O.), sodium polyoxyethylene alkyl (12 to 14) ether sulfate (3 E.O.), sodium polyoxyethylene alkyl (12 to 15) ether sulfate (3 E.O.), disodium polyoxyethylene alkyl (12 to 14) sulfosuccinate (7 E.O.), sodium polyoxyethylene undecyl ether sulfate, liquid sodium polyoxyethylene octyl phenyl ether sulfate, ammonium polyoxyethylene oleyl ether sulfate, disodium polyoxyethylene lauryl sulfosuccinate, sodium polyoxyethylene nonyl phenyl ether sulfate, sodium polyoxyethylene pentadecyl ether sulfate, triethanolamine polyoxyethylene myristyl ether sulfate, sodium polyoxyethylene myristyl ether sulfate, sodium polyoxyethylene myristyl ether sulfate (3 E.O.), liquid sodium polyoxyethylene lauryl ether acetate (16 E.O.), ammonium polyoxyethylene lauryl ether sulfate (2 E.O.), triethanolamine polyoxyethylene lauryl ether sulfate, sodium polyoxyethylene lauryl ether sulfate, diethanolamine myristyl sulfate, sodium myristyl sulfate, potassium myristyl sulfate, sodium N-myristoyl-L-glutamate, sodium myristoylmethylaminoacetate, liquid myristoyl methyl-β-alanine sodium salt, myristoyl methyltaurine sodium salt, medicinal soaps, triethanolamine/magnesium coco alkyl sulfate, triethanolamine N-coconut oil fatty-acyl-L-glutamate, sodium N-coconut oil fatty-acyl-L-glutamate, sodium coconut oil fatty acid ethyl ester sulfonate, coconut oil fatty acid potassium salt, liquid coconut oil fatty acid potassium salt, sodium N-coconut oil fatty/hydrogenated fatty-acyl-L-glutamate, coconut oil fatty acid sarcosine, coconut oil fatty acid sarcosine triethanolamine salt, coconut oil fatty acid sarcosine sodium salt, coconut oil fatty acid triethanolamine salt, liquid triethanolamine salt of coconut oil fatty acid, coconut oil fatty acid sodium salt, coconut oil fatty acid methyl alanine sodium salt, liquid coconut oil fatty acid methyl alanine sodium salt, coconut oil fatty acid methyltaurine potassium salt, coconut oil fatty acid methyltaurine sodium salt, sodium laurylamino dipropionate, liquid sodium laurylamino dipropionate (30%), sodium lauryl sulfoacetate; sodium lauryl benzenesulfonate, lauryl sulfate, ammonium lauryl sulate, potassium lauryl sulfate, diethanolamine lauryl sulfate, triethanolamine lauryl sulfate, sodium lauryl sulfate, magnesium lauryl sulfate, monoethanolainine lauryl sulfate, potassium laurate, lauric acid triethanolamine, liquid lauric acid triethanolamine, sodium laurate, lauric acid/myristic acid triethanolamine, lauroyl-L-glutamic acid triethanolamine, sodium N-lauroyl-L-glutamate, lauroyl sarcosine, lauroyl sarcosine potassium, liquid lauroyl sarcosine triethanolamine salt, lauroyl sarcosine sodium, liquid lauroyl methyl-β-alanine sodium salt, lauroyl methyltaurine sodium salt, and liquid lauroyl methyltaurine sodium salt.

Specific examples of pH-regulating agents include hydrochloric acid, sulfuric acid, phosphoric acid, ammonium hydrogenphosphate, disodium hydrogenphosphate, dipotassium hydrogenphosphate, ammonium dihydrogenphosphate, sodium dihydrogenphosphate, potassium dihydrogenphosphate, trisodium phosphate, tripotassium phosphate, acetic acid, ammonium acetate, sodium acetate, potassium acetate, citric acid, sodium citrate, diammonium citrate, sodium carbonate, potassium carbonate, ammonium carbonate, sodium hydrogencarbonate, ammonium hydrogencarbonate, sodium hydroxide, potassium hydroxide, ammonia, and triethanolamine.

Specific examples of antiseptics, mildew-proofing agents, and rust preventives include benzoic acid, aluminum benzoate, sodium benzoate, isopropyl methyl phenol, ethylhexanediol, lysozyme chloride, chlorhexydine chloride, octylphenoxyethanol, ortho-phenylphenol, sodium perborate, photosensitive material No. 101, photosensitive material No. 201, photosensitive material No. 301, photosensitive material No. 401, liquid chlorhexydine gluconate, cresol, chloramine T, chlorxylenol, chlorcresol, chlorphenesin, chlorhexydine, chlorobutanol, resorcin acetate, salicylic acid, sodium salicylate, domiphen bromide, zinc pyrithion, liquid zinc pyrithion, sorbic acid, potassium sorbate, thianthol, thioxolone, thimol, chiram, dehydroacetic acid, sodium dehydroacetate, trichlorocarbanilide, trichlorohydroxy diphenyl ether, isobutyl paraoxybenzoate, isopropyl paraoxybenzoate, ethyl paraoxybenzoate, butyl paraoxybenzoate, propyl paraoxybenzoate, benzyl paraoxybenzoate, methyl paraoxybenzoate, sodium methyl paraoxybenzoate, parachlorphenol, sodium paraphenol sulfonate (dihydrate), halocarbane, phenoxyethanol, phenol, hexachlorophane, mononitroguaiacol, mononitroguaiacol sodium, paradimethylaminostyrylheptylmethyl lyazolinium iodide, lauryltrimethylammonium trichlorophenoxide, oxyquinoline sulfate, oxyquinoline phosphate, and resorcin.

Next, explanations are provided regarding the raw material for cosmetics of the present invention. The raw material for cosmetics of the present invention consists of the above-described micro-emulsion composition. However, so long as the effects of the present invention are not impaired, other components known as additives for cosmetic raw materials consisting of silicone emulsions can be added and combined therewith in order to further improve its stability in mixtures with cosmetics. The above-described anionic surface active agents, nonionic surface active agents, pH-regulating agents, antiseptics, mildew-proofing agents, and rust preventives are suggested as such additives. These components can be used singly or as a combination of several components.

Adding the following various raw materials to the raw material for cosmetics of the present invention makes it possible to obtain skin cosmetics exhibiting excellent compatibility with the skin and imparting it with superior moisturized feel and slip. In addition to the above-mentioned anionic surface active agents, nonionic surface active agents, pH-regulating agents, antiseptics, mildew-proofing agents, rust preventives; the raw materials for skin cosmetics are exemplified by avocado oil, almond oil, olive oil, cacao butter, sesame oil, wheat germ oil, safflower oil, shea butter, turtle oil, tung oil, persic oil, castor oil, grapeseed oil, macadamia nut oil, mink oil, egg yolk oil, Japan wax, coconut oil, rose hip oil, hydrogenated oils and other oils and fats; orange roughy oil, carnauba wax, candelilla wax, spermaceti wax, jojoba oil, montan wax, beeswax, lanolin, and other waxes; liquid paraffin, vaseline, paraffin, ceresin, microcrystalline wax, squalane and other hydrocarbons; lauric acid, myristic acid, palmitic acid, stearic acid, oleic acid, behenic acid, undecylenic acid, oxystearic acid, linoleic acid, lanolinic acid, synthetic fatty acids, and other higher fatty acids; ethyl alcohol, isopropyl alcohol, lauryl alcohol, cetyl alcohol, cetostearyl alcohol, stearyl alcohol, oleyl alcohol, behenyl alcohol, lanolin alcohol, hydrogenated lanolin alcohol, hexyldecanol, octyl dodecanol, isostearyl alcohol, and other alcohols; cholesterol, dihydrocholesterol, phytosterol, and other sterols; ethyl linoleate, isopropyl myristate, lanolin fatty acid isopropyl, hexyl laurate, myristyl myristate, cetyl myristate, octyldodecyl myristate, decyl oleate, octyldodecyl oleate, hexyldecyl dimethyloctanoate, cetyl isooctanoate, cetyl palmitate, glycerin trimyristate, glycerin tri(capryl caprate), propylene glycol dioleate, glycerin triisostearate, glycerin triisooctanoate, cetyl lactate, myristyl lactate, diisostearyl malate, and other fatty acid esters; glycerin, propylene glycol, 1,3-butylene glycol, polyethylene glycol, sodium d, 1-pyrrolidonecarboxylate, sodium lactate, sorbitol, sodium hyaluronate, and other humectants; cationic surface active agents; betaine-type, amino acid-type, imidazoline-type, lecithin and other amphoteric surface active agents; iron oxides and other colored pigments; zinc oxide, titanium oxide, zirconium oxide, and other white pigments; mica, talc, sericite, and other skin-color pigments, and other pigments; dimethylpolysiloxane, methylphenylpolysiloxane, octamethyltetracyclosiloxane, decamethylcyclopentasiloxane, polyether-modified silicone oils, amino-modified silicone oils, and other silicone oils; demineralized water; carrageenan, alginic acid, gum arabic, traganth, pectin, starch, xanthan gum, polyvinyl alcohol, polyvinyl pyrrolidone, sodium polyacrylate, polyethylene glycol, and other thickening agents; silicone-acrylic copolymers, silicone resins, acrylic polymers and other film-forming agents; and, furthermore, UV-absorbers, anti-bacterial agents, anti-inflammatory agents, anti-perspirants, perfume, anti-oxidants, and blowing agents. In addition, hand creams, skin creams, foundations, eye shadow, face wash, and body shampoos are specifically suggested as skin cosmetic materials.

In addition, when the raw material for cosmetics of the present invention is used in hair cosmetic materials, combining it with film-forming agents, anti-freeze agents, oils, emulsifying agents, moisturizing agents, anti-dandruff agents, anti-oxidants, chelating agents, UV-absorbers, perfumes, colorants and various other raw materials in addition to the above-describe anionic surface active agents, nonionic surface active agents, pH-adjusting agents, antiseptics, mildew-proofing agents, and rust preventives, makes it possible to obtain hair cosmetic materials that are excellent in adhesion to hair and can give hair superior moisturized feel and slip. The film-forming agents are specifically exemplified by non-functional silicone resins and silicone resins modified with amino groups and organic groups containing fluorine, poly(N-methylpyrrolidone), poly(N-acylalkyleneimine), and copolymers of silicone compounds and polymers of (meth)acrylic radical-polymerizable monomers. There are no particular limitations concerning the anti-freeze agents, with typical agents including ethanol, isopropyl alcohol, 1,3-butylene glycol, ethylene glycol, propylene glycol, and glycerin. The oils can be any compounds typically used in cosmetics, whose representative examples include microcrystalline wax, paraffin wax, spermaceti wax, beeswax, Japan wax, sugar cane wax, and other waxes, or their mixtures; liquid paraffin, α-olefin oligomers, squalane, squalene, and other hydrocarbon oils or their mixtures; cetanol, stearyl alcohol, isostearyl alcohol, hardened castor oil-derived alcohols, behenyl alcohol, lanolin alcohol, and other linear or branched saturated or unsaturated, unsubstituted or hydroxy-substituted higher alcohols or their mixtures; palmitic acid, myristic acid, oleic acid, stearic acid, hydroxystearic acid, isostearic acid, behenic acid, castor oil fatty acid, coconut oil fatty acid, tallow fatty acid, and other linear or branched saturated or unsaturated unsubstituted or hydroxy-substituted higher fatty acids or their mixtures; olive oil, coconut oil, rapeseed oil, palm oil, castor oil, hardened castor oil, peanut oil, tallow, hydrogenated tallow, jojoba oil, hardened jojoba oil, monostearic acid glyceride, monooleic acid glyceride, dipalmitic acid glyceride, trimyristic acid glyceride, oleyl oleate, isostearyl isostearate, palmityl behenate, isopropyl palmitate, stearyl acetate, dihydroxystearic acid ester and other esters; linear, branched, or cyclic low molecular weight silicone oils, amino-modified silicone oils, fatty acid-modified silicone oils, alcohol-modified silicone oils, polyether-modified silicone oils, phosphoric acid (phosphate)-containing silicone oils, sulfuric acid (sulfate)-containing silicone oils, fluorine-modified alkyl-containing silicone oils, alkyl-modified silicone oils, epoxy-modified silicone oils, and other silicone oils, high molecular silicones; silicone resins that are solvent-soluble, possess thermoplastic properties or are liquid or rubbery at room temperature, or their mixtures. These silicones are preferably latex-like, with conventional commonly used agents such as, for example, glycerin monostearate, sorbitan monopalmitate, polyoxyethylene cetyl ether, polyoxyethylene stearic acid ether, and polyoxyethylene sorbitan monolaurate suggested as emulsifying agents to be used therefor. The humectants are exemplified by hexylene glycol, polyethylene glycol 600, sodium pyroglutamate, and glycerin. The anti-dandruff agents are exemplified by iodine, selenium sulfide, zinc pyridium-1-thiol-N-oxide, salicylic acid, 2,4,4′-trichloro-2′-hydroxydiphenyl ether, 1-hydroxy-2-pyridone compounds. BHA, BHT, and γ-oryzanol are suggested as the anti-oxidants. The chelating agents are exemplified by ethylenediamine tetraacetate, citric acid, ethane-1-hydroxy-1,1-diphosphonic acid and their salts. The UV-absorbers are exemplified by benzophenone derivatives represented by 2-hydroxy-4-methoxybenzophenone, benzotriazole derivatives represented by 2-(2′-hydroxy-5′-methylphenyl)benzotriazole, and cinnamic acid esters. Furthermore, preferable compounds include glycerin, propylene glycol, dipropylene glycol, 1,3-butylene glycol, and other polyhydric alcohols, monoalkyltrimethylammonium salts, dialkyldimethylammonium salts, and other quaternary ammonium salts, specifically, stearyltrimethylammonium chloride, behenyltrimethylammonium chloride, distearyldimethylammonium chloride, dibehenyidimethylammonium chloride, and other cationic surface active agents, or amphoteric surface active agents, squalane, lanolin, perfluoropolyether, cationic polymers, and other sensitivity improvers, propylene glycol, glycerin, sorbitol, and other humectants, methylcellulose, carboxyvinyl polymers, hydroxyethylcellulose, polyoxyethylene glycol distearate, ethanol, and other viscosity-adjusting agents, pearlescing agents, perfumes, colorants, dyes, blowing agents, vitamins, hair-nourishing agents, hormones, and other chemicals, triclosan, trichlorocarban, and other anti-bacterial agents, potassium glycyrrhyzate, tocopherol acetate, and other anti-inflammatory agents, zinc pyrithione, octopyrox, and other anti-dandruff agents, methylparabene, butylparabene, and other antiseptics, atomizing agents, and other components listed in the Encyclopedia of Shampoo Ingredients (Micelle press, 1985). In addition, shampoo, rinse-in shampoos, hair conditioners, hair treatment products, hair setting lotions, blow styling agents, hair spray, foaming styling agents, gel styling agents, hair cream, preparations for hair growth, hair nourishing formulations, and hair dyeing agents are specifically suggested as the hair cosmetics.

### BEST MODE FOR CARRYING OUT THE INVENTION

Examples. Hereinbelow the present invention is explained in detail by referring to examples. In the examples, the word “part(s)” stands for “part(s) by weight” and the symbol “%” stands for “wt %.” In addition, the measurement of the physical properties of the obtained emulsions and the evaluation of hair treated therewith was carried out in accordance with the following methods.

Average Particle Size of Emulsion

The average particle size was measured using a “Coulter Model N4” from Coulter Electronics, Inc. (United States).

Number Average Molecular Weight of Polyorganosiloxane

The number average molecular weight, converted to polystyrene, was measured using a GPC analyzer (from Shimazu Corp.; S-8120) by causing an emulsion to undergo complete separation by adding alcohol thereto and then dissolving the oily component in a solvent.

Dry Residue of Polyorganosiloxane

A precisely weighed amount of a polyorganosiloxane micro-emulsion was placed in an aluminum cup and left stand in an oven at 105° C. for 2 hours, whereupon the dry residue (%) was calculated.

Dry residue (%)=(weight of emulsion after drying/weight of emulsion prior to drying)×100

External Appearance of Emulsion

Immediately after preparation, the external appearance of the emulsion was evaluated in the following manner.

◯: Colorless transparent liquid.

Δ: Light yellow˜yellow transparent liquid.

×: Milky-white liquid.

Stability

A sample was left stand for 30 days in an environment tester which was set to go through a temperature cycle from 0° C. to 50° C. every 12 hours. The transparency, stability in mixtures, and color stability of the sample after 30 days and immediately after preparation were visually observed and evaluated using the following 3-point scale.

∘Transparency

◯: Transparent

Δ: Slight turbidity noticed.

×: Turbidity was clearly seen.

∘Stability in mixtures

◯: No separation noticed.

Δ: Slight separation noticed.

×: Conspicuous separation noticed.

∘ Color stability

◯: No changes in color tone noticed.

Δ: Slight changes in color tone noticed.

×: Conspicuous changes in color tone noticed.

Hair Shampoo Evaluation Method

As pre-treatment of the hair to be treated, a bundle of hair with a weight of 5 g was washed in a 10 wt % aqueous solution of sodium polyoxyethylene (4) lauryl sulfate, whereupon the hair was rinsed with running water and naturally dried for 24 hours or more. The pre-treated hair was immersed in a hair shampoo composition for 10 seconds and thoroughly washed with water. After that, the hair was brushed until the individual strands did not cling to each other and then naturally dried for at least 24 hours. The resultant shampoo-treated hair was subjected to organoleptic evaluation by 30 panelists.

Body Shampoo Evaluation Method

The skin of 30 panelists was washed with a body shampoo composition for 30 seconds and then rinsed with running water. After completely removing moisture with a towel, organoleptic evaluation was carried out.

### EXAMPLE 1

After dissolving 1 parts of polyoxyethylene lauryl ether (25 EO) in 15 parts of ion exchange water, 20 parts of octamethylcyclotetrasiloxane was added and premixed with the solution. A coarse emulsion with an average particle size of 0.25 μm was obtained by passing the mixture twice through a homogenizer under a pressure of 350 kg/cm². After mixing 9 parts of N-lauroylmethyltaurine sodium salt, 23 parts of ion exchange water, 6 parts of hydrochloric acid, and 3.5 parts of polyoxyethylene lauryl ether (25 EO) in a separate container, the mixture was maintained at 70° C. Next, the above-described coarse emulsion was added to this mixture under agitation in a dropwise manner over a period of 2 hours and the resultant mixture was maintained for 8 hours at 70° C. The mixture was cooled to 25° C. and maintained at this temperature for another 10 hours. After that a polydimethylsiloxane micro-emulsion (Sample A-1) was obtained by terminating the polymerization reaction by adding a 10% aqueous solution of sodium hydroxide to the mixture in a dropwise manner until the pH of the reaction solution became close to 7. The external appearance and physical properties of the resultant micro-emulsion are listed in Table 2.

### EXAMPLE 2

After dissolving 1 part of polyoxyethylene lauryl ether (25 EO) in 15 parts of ion exchange water, a mixture of 19.9 parts of octamethylcyclotetrasiloxane and 0.1 parts of methyltrimethoxysilane was added and premixed with the solution. A coarse emulsion with an average particle size of 0.25 μm was obtained by passing the mixture twice through a homogenizer under a pressure of 350 kg/cm². After mixing 9 parts of N-lauroylmethyltaurine sodium salt, 23 parts of ion exchange water, 6 parts of hydrochloric acid, and 3.5 parts of polyoxyethylene lauryl ether (25 EO) in a separate container, the mixture was maintained at 70° C. Next, the above-described coarse emulsion was added to this mixture under agitation in a dropwise manner over a period of 2 hours and the resultant mixture was maintained for 8 hours at 70° C. The mixture was cooled to 25° C. and maintained at this temperature for another 10 hours. After that, a polydimethylsiloxane micro-emulsion (Sample A-2) was obtained by terminating the polymerization reaction by adding a 10% aqueous solution of sodium hydroxide to the mixture in a dropwise manner until the pH of the reaction solution became close to 7. The external appearance and physical properties of the resultant micro-emulsion are listed in Table 2.

### EXAMPLE 3

After dissolving 1 part of polyoxyethylene lauryl ether (25 EO) in 15 parts of ion exchange water, a mixture of 19.95 parts of octamethylcyclotetrasiloxane and 0.05 parts of hexamethyldisiloxane were added and premixed with the solution. A coarse emulsion with an average particle size of 0.25 μm was obtained by passing the mixture twice through a homogenizer under a pressure of 350 kg/cm². After mixing 9 parts of N-lauroylmethyltaurine sodium salt, 23 parts of ion exchange water, 6 parts of hydrochloric acid, and 3.5 parts of polyoxyethylene lauryl ether (25 EO) in a separate container, the mixture was maintained at 70° C. Next, the above-described coarse emulsion was added to this mixture under agitation in a dropwise manner over a period of 2 hours and the resultant mixture was maintained for 8 hours at 70° C. The mixture was cooled to 55° C. and maintained at this temperature for another 10 hours. After that, a polydimethylsiloxane micro-emulsion (Sample A-3) was obtained by terminating the polymerization reaction by adding a 10% aqueous solution of sodium hydroxide to the mixture in a dropwise manner until the pH of the reaction solution became close to 7. The external appearance and physical properties of the resultant micro-emulsion are listed in Table 2.

### EXAMPLE 4

After mixing 9 parts of N-lauroylmethyltaurine sodium salt, 38 parts of ion exchange water, 6 parts of hydrochloric acid, and 4.5 parts of polyoxyethylene lauryl ether (25 EO), the mixture was maintained at 70° C. Next, 20 parts of octamethylcyclotetrasiloxane were added to this mixture under agitation in a dropwise manner over a period of 2 hours and the resultant mixture was maintained for 8 hours at 70° C. The mixture was cooled to 25° C. and maintained at this temperature for another 10 hours. After that, a polydimethylsiloxane micro-emulsion (Sample A-4) was obtained by terminating the polymerization reaction by adding a 10% aqueous solution of sodium hydroxide to the mixture in a dropwise manner until the pH of the reaction solution became close to 7. The external appearance and physical properties of the resultant micro-emulsion are listed in Table 2.

### EXAMPLE 5

After mixing 9 parts of N-lauroylmethyltaurine sodium salt, 38 parts of ion exchange water, 6 parts of hydrochloric acid, and 4.5 parts of polyoxyethylene lauryl ether (25 EO), the mixture was maintained at 70° C. Next, a mixture of 19.9 parts of octamethylcyclotetrasiloxane and 0.1 part of γ-glycidoxypropyltrimethoxysilane was added to this mixture under agitation in a dropwise manner over a period of 2 hours and the resultant mixture was maintained for 8 hours at 70° C. The mixture was cooled to 55° C. and maintained at this temperature for another 10 hours. After that, a polydimethylsiloxane micro-emulsion (Sample A-5) was obtained by terminating the polymerization reaction by adding a 10% aqueous solution of sodium hydroxide to the mixture in a dropwise manner until the pH of the reaction solution became close to 7. The external appearance and physical properties of the resultant micro-emulsion are listed in Table 2.

### Comparative Example 1

After dissolving 2 parts of dodecylbenzenesulfonic acid in 52 parts of ion exchange water, 40 parts of octamethylcyclotetrasiloxane were added and premixed with the solution. A coarse emulsion with an average particle size of 0.26 μm was obtained by passing the mixture twice through a homogenizer under a pressure of 350 kg/cm². After maintaining the resultant coarse emulsion at 85° C. to 90° C. for 4 hours, the emulsion was cooled to 45° C. and maintained at this temperature for another 5 hours. After that, a polydimethylsiloxane emulsion (Sample B-1) was obtained by terminating the polymerization reaction by adding a 10% aqueous solution of sodium hydroxide in a dropwise manner until the pH of the emulsion became close to 7. The external appearance and physical properties of the resultant emulsion are listed in Table 2.

Comparative Example 2. After dissolving 1 part of polyoxyethylene lauryl ether (25 EO) in 15 parts of ion exchange water, 20 parts of octamethylcyclotetrasiloxane were added and premixed with the solution. A coarse emulsion with an average particle size of 0.25 μm was obtained by passing the mixture twice through a homogenizer under a pressure of 350 kg/cm². After mixing 9 parts of dodecylbenzenesulfonic acid, 29 parts of ion exchange water, and 3.5 parts of polyoxyethylene lauryl ether (25 EO) in a separate container, the mixture was maintained at 70° C. Next, the above-described coarse emulsion was added to this mixture under agitation in a dropwise manner over a period of 2 hours and the resultant mixture was maintained for 8 hours at 70° C. The mixture was cooled to 25° C. and maintained at this temperature for another 10 hours. After that, a polydimethylsiloxane emulsion (Sample B-2) was obtained by terminating the polymerization reaction by adding a 10% aqueous solution of sodium hydroxide in a dropwise manner until the pH of the reaction solution became close to 7. The external appearance and physical properties of the resultant emulsion are listed in Table 2.

### Comparative Example 3

After mixing 9 parts of an anionic surface active agent, which was a mixture of 75% sodium tetradecenesulfonate and 25% of sodium hydroxytetradecanesulfonate, 38 parts of ion exchange water, 4.5 parts of polyoxyethylene lauryl ether (25 EO), and 6 parts of hydrochloric acid, the mixture was maintained at 70° C. Next, 20 parts of octamethylcyclotetrasiloxane were added to this mixture under agitation in a dropwise manner over a period of 2 hours and the resultant mixture was maintained for 8 hours at 70° C. The mixture was cooled to 25° C. and maintained at this temperature for another 10 hours. After that, a polydimethylsiloxane emulsion (Sample B-3) was obtained by terminating the polymerization reaction by adding a 10% aqueous solution of sodium hydroxide in a dropwise manner until the pH of the reaction solution became close to 7. The external appearance and physical properties of the resultant emulsion are listed in Table 2.

TABLE 1

| | Sample | Sample | Sample | Sample | Sample | | Sample | Sample |
|---|---|---|---|---|---|---|---|---|
| | A-1 | A-2 | A-3 | A-4 | A-5 | Sample | B-2 | B-3 |
| | (*1) | (*1) | (*1) | (*2) | (*2) | B-1 | (*1) | (*2) |
| N-lauroylmethyltaurine | 9.0 | 9.0 | 9.0 | 9.0 | 9.0 | — | — | — |
| sodium salt | | | | | | | | |
| Dodecylbenzenesulfonic | — | — | — | — | — | 2.0 | 9.0 | — |
| acid | | | | | | | | |
| Mixture of sodium | — | — | — | — | — | — | — | 9.0 |
| tetradecenesulfonate | | | | | | | | |
| and sodium hydroxytetradecanesulfonate | | | | | | | | |
| Octamethylcyclotetrasiloxane | 20.0 | 19.9 | 19.95 | 20.0 | 19.9 | 40.0 | 20.0 | 20.0 |
| Methyltrimethoxysilane | — | 0.1 | — | — | — | — | — | — |
| Hexamethyldisiloxane | — | — | 0.05 | — | — | — | — | — |
| γ-glycidoxypropyltrimethoxysilane | — | — | — | — | 0.1 | — | — | — |
| Ion exchange water | 38.0 | 38.0 | 38.0 | 38.0 | 38.0 | 52.0 | 44.0 | 38.0 |
| Polyoxyethylene lauryl | 4.5 | 4.5 | 4.5 | 4.5 | 4.5 | — | 4.5 | 4.5 |
| ether (25EO) | | | | | | | | |
| Hydrochloric acid | 6.0 | 6.0 | 6.0 | 6.0 | 6.0 | — | — | 6.0 |
| 10% aqueous solution | Appr. | Appr. | Appr. | Appr. | Appr. | Appr. | Appr. | Appr. |
| of sodium hydroxide | amount | amount | amount | amount | amount | Amount | amount | Amount |

(*1) Emulsion prepared based on the method (see Japanese Patent Application Publication(Kokai) No. Sho 62(1987)-141029), in which a coarse emulsion consisting of water, surface active agent, and cyclic organosiloxane is added to a mixture of water, polymerization catalyst, and surface active agent.
(*2) Emulsion prepared based on the method (see Japanese Patent Application Publication(Kokai) No. Hei 10(1998)-265577), in which cyclic organosiloxane is added to a mixture of ionic surface active agent, polymerization catalyst, and water under agitation.

TABLE 2

| | | Average | Number average | Dry | | |
|---|---|---|---|---|---|---|
| | | particle | molecular weight | residue | External | Evaluation of micro- |
| | | size (μm) | of siloxane | (%) | appearance | emulsion |
| Examples | Sample A-1 | 0.04 | 68500 | 34.8 | Colorless, | ◯ |
| | | | | | transparent liquid | Excellent |
| | Sample A-2 | 0.04 | 107900 | 35.5 | Colorless, | ◯ |
| | | | | | transparent liquid | Excellent |
| | Sample A-3 | 0.04 | 28000 | 35.4 | Colorless, | ◯ |
| | | | | | transparent liquid | Excellent |
| | Sample A-4 | 0.04 | 69200 | 34.7 | Colorless, | ◯ |
| | | | | | transparent liquid | Excellent |
| | Sample A-5 | 0.04 | 109000 | 35.2 | Colorless, | ◯ |
| | | | | | transparent liquid | Excellent |
| Comparative | Sample B-1 | 0.23 | 67200 | 38.7 | Milky-white liquid | X |
| Examples | | | | | | Not a micro-emulsion |
| | Sample B-2 | 0.04 | 70500 | 33.5 | Yellow transparent | Δ |
| | | | | | liquid | Discoloration |
| | Sample B-3 | 0.04 | 68300 | 34.5 | Yellow transparent | Δ |
| | | | | | liquid | Discoloration |

### EXAMPLE 6

A hair shampoo composition was prepared by combining Sample A-1 prepared in Example 1 with a hair shampoo base consisting of the ingredients listed hereinbelow in such a manner that the concentration of the polydimethylsiloxane in the mixture was 1 wt %. The stability of the resultant hair shampoo composition was measured. In addition, hair was washed using the hair shampoo composition, and an organoleptic evaluation was carried out after treatment. Furthermore, the hair was subjected to visual evaluation to determine the degree of its glossiness.

Hair shampoo compositions were prepared and hair treatment and evaluation were conducted in the same manner using Samples A-2 to A-5 prepared in Examples 2 to 5. The results are listed in Table 3.

◯ Hair shampoo base

| | | |
|---|---|---|
| 25% aqueous solution of N-lauroylmethyltaurine sodium | 20.0 | parts |
| Lauroylsarcosine sodium | 6.0 | parts |
| 25% aqueous solution of lauryldimethylbetaine | 10.0 | parts |
| Coconut oil fatty acid diethanolamide | 4.0 | parts |
| Propylene glycol | 5.0 | parts |
| Phenoxyethanol | 1.0 | parts |
| (2-hydroxy-3- | 0.5 | parts |
| (trimethylammonio)propyl)hydroxyethylcellulose-o-chloride | | |
| Ion exchange water | 48.0 | parts |

### Comparative Example 4

Hair shampoo compositions were prepared in the same manner as in Example 6 using Samples B-1 to B-3 prepared in Comparative Examples 1 to 3. Evaluation of the obtained hair shampoo compositions was conducted in the same manner as in Example 6 and the results are listed in Table 3.

TABLE 3

| | | | Comparative | | | | | | | |
|---|---|---|---|---|---|---|---|---|---|---|
| | | | Example 6 | | | | | Example 4 | | |
| No. of sample used | | | A-1 | A-2 | A-3 | A-4 | A-5 | B-1 | B-2 | B-3 |
| Results | Transparency | Immediately | ∘ | ∘ | ∘ | ∘ | ∘ | x | ∘ | ∘ |
| | | after preparation | | | | | | | | |
| | | After 50° C. | ∘ | ∘ | ∘ | ∘ | ∘ | x | ∘ | ∘ |
| | | cycle test | | | | | | | | |
| | Stability in | Immediately | ∘ | ∘ | ∘ | ∘ | ∘ | ∘ | ∘ | ∘ |
| | mixtures | after preparation | | | | | | | | |
| | | After 50° C. | ∘ | ∘ | ∘ | ∘ | ∘ | x | Δ | Δ |
| | | cycle test | | | | | | | | |
| | Color tone | Immediately | ∘ | ∘ | ∘ | ∘ | ∘ | ∘ | ∘ | ∘ |
| | stability | after preparation | | | | | | | | |
| | | After 50° C. | ∘ | ∘ | ∘ | ∘ | ∘ | x | Δ | Δ |
| | | cycle test | | | | | | | | |
| | Organoleptic | Excellent feel | 30 | 29 | 28 | 29 | 28 | 25 | 25 | 28 |
| | evaluation | Somewhat | 0 | 1 | 2 | 1 | 2 | 3 | 3 | 2 |
| | (people) | inferior slip | | | | | | | | |
| | | lnferior slip | 0 | 0 | 0 | 0 | 0 | 2 | 2 | 0 |
| | Gloss | There is gloss | 29 | 30 | 29 | 29 | 28 | 23 | 24 | 28 |
| | (people) | Slightly inferior | 1 | 0 | 1 | 1 | 2 | 5 | 3 | 2 |
| | | gloss | | | | | | | | |
| | | Inferior gloss | 0 | 0 | 0 | 0 | 0 | 2 | 3 | 0 |

### EXAMPLE 7

A body shampoo composition was prepared by combining Sample A-1 prepared in Example 1 with a body shampoo base consisting of the ingredients listed hereinbelow in such a manner that the concentration of the polydimethylsiloxane in the mixture was 2 wt %. The stability of the resultant body shampoo composition was measured. In addition, the skin was washed using the body shampoo composition and subjected to organoleptic evaluation.

In addition, body shampoo compositions were prepared and skin washing and evaluation were conducted in the same manner as described above using Samples A-2 to A-5 prepared in Examples 2 to 5. The results are listed in Table 4.

◯ Body shampoo base

| | |
|---|---|
| 30% aqueous solution of lauroylsarcosine sodium | 15.0 parts |
| 27% aqueous solution of disodium lauryl sulfosuccinate | 15.0 parts |
| Ion exchange water | 60.0 parts |

### Comparative Example 5

Body shampoo compositions were prepared in the same manner as in Example 7 using Samples B-1 to B-3 prepared in Comparative Examples 1 to 3. Evaluation of the obtained body shampoo compositions was conducted in the same manner as in Example 7 and the results are listed in Table 4.

TABLE 4

| | | | Comparative | | | | | | | |
|---|---|---|---|---|---|---|---|---|---|---|
| | | | Example 7 | | | | | Example 5 | | |
| No. of sample used | | | A-1 | A-2 | A-3 | A-4 | A-5 | B-1 | B-2 | B-3 |
| Results | Transparency | Immediately | ∘ | ∘ | ∘ | ∘ | ∘ | x | ∘ | ∘ |
| | | after preparation | | | | | | | | |
| | | After 50° C. | ∘ | ∘ | ∘ | ∘ | ∘ | x | ∘ | ∘ |
| | | cycle test | | | | | | | | |
| | Stability in | Immediately | ∘ | ∘ | ∘ | ∘ | ∘ | x | ∘ | ∘ |
| | mixtures | after preparation | | | | | | | | |
| | | After 50° C. | ∘ | ∘ | ∘ | ∘ | ∘ | x | Δ | Δ |
| | | cycle test | | | | | | | | |
| | Color tone | Immediately | ∘ | ∘ | ∘ | ∘ | ∘ | x | ∘ | ∘ |
| | stability | after preparation | | | | | | | | |
| | | After 50° C. | ∘ | ∘ | ∘ | ∘ | ∘ | x | x | x |
| | | cycle test | | | | | | | | |
| | Skin feel | Excellent feel | 29 | 29 | 28 | 28 | 29 | 24 | 23 | 28 |
| | (people) | Slightly inferior | 1 | 1 | 2 | 2 | 1 | 2 | 2 | 2 |
| | | slip | | | | | | | | |
| | | Inferior slip | 0 | 0 | 0 | 0 | 0 | 4 | 5 | 0 |

### INDUSTRIAL APPLICABILITY

Because the present polyorganosiloxane micro-emulsion composition as described above makes use of N-acylalkyltaurine and/or its salts as surface active agents, it is colorless and transparent and possesses superior cosmetic performance in terms of imparting moisturized feel and slip and low irritation to the skin and scalp. In addition, its advantages include superior stability of the emulsion, silicone stability, and stability in mixtures with various cosmetic materials, as well as high color tone stability with extremely low discoloration over time. For this reason, the micro-emulsion composition of the present invention is useful as a component added to raw materials for skin cosmetics and hair cosmetics. In other words, it is not a cosmetic by itself, but it is suitable as a raw material used for compounding with other ingredients and is characterized by the fact that when it is used as a raw material for cosmetics, cosmetics of excellent skin feel are obtained.

What is claimed is:

1. A polyorganosiloxane micro-emulsion composition comprising (A) a polyorganosiloxane, (B) a material selected from the group consisting of (i) N-acylalkyltaurine and (ii) an N-acylalkyltaurine salt, and (C) water, wherein the polyorganosiloxane micro-emulsion composition has an average particle size of emulsion particles of less than 0.15 μm.

2. The polyorganosiloxane micro-emulsion composition as claimed in claim 1 further containing (D) a nonionic surface active agent.

3. The polyorganosiloxane micro-emulsion composition as claimed in claim 2 comprising (A) 100 parts by weight of a polyorganosiloxane, (B) 5 to 300 parts by weight of a material selected from the group consisting of (i) N-acylalkyltaurine and (ii) an N-acylalkyltaurine salt, (C) 10 to 1000 parts by weight of water, and (D) 5 to 200 parts by weight of a nonionic surface active agent.

4. The polyorganosiloxane micro-emulsion composition as claimed in claim 2 that is obtained by subjecting a polyorganosiloxane of a lower molecular weight than component (A) to emulsion polymerization in water in the presence of (B) a material selected from the group consisting of (i) N-acylalkyltaurine and (ii) an N-acylalkyltaurine salt and (D) a nonionic surface active agent.

5. The polyorganosiloxane micro-emulsion composition as claimed in claim 3 that is obtained by subjecting a polyorganosiloxane of a lower molecular weight than component (A) to emulsion polymerization in water in the presence of (B) a material selected from the group consisting of (i) N-acylalkyltaurine and (ii) an N-acylalkyltaurine salt and (D) a nonionic surface active agent.

6. The polyorganosiloxane micro-emulsion composition as claimed in claim 1, wherein the number average molecular weight of component (A) at 25° C. is in the range of 1,000 to 1,000,000.

7. The polyorganosiloxane micro-emulsion composition as claimed in claim 1, wherein component (A) is described by the average unit formula R¹ₙSiO_{(4-n/2)} wherein each R¹ is independently selected from the group consisting of substituted and unsubstituted hydrocarbon groups and n is a number greater than 0 and less than 4.

8. The polyorganosiloxane micro-emulsion composition as claimed in claim 6, where n has a value between 1.8 and 2.2.

9. The polyorganosiloxane micro-emulsion composition as claimed in claim 1, where component (B) is selected from the group consisting of (i) N-acylalkyltaurine and (ii) a salt thereof described by the formula R⁵CONR⁶CH₂CH₂SO₃M wherein each R⁵ and R⁶ are independently selected from the group consisting of substituted and unsubstituted monovalent hydrocarbon groups and M is selected from the group consisting of the hydrogen atom, alkali metal atom, ammonium, and triethanolammonium.

10. The polyorganosiloxane micro-emulsion composition as claimed in claim 8, wherein R⁵ comprises 1 to 30 carbon atoms and R⁶ comprises 1 to 6 carbon atoms.

11. The polyorganosiloxane micro-emulsion composition as claimed in claim 8, wherein component (B) is selected from the group consisting of N-lauroyl methyltaurine sodium salt, N-coconut oil fatty acid methyltaurine potassium salt, N-coconut oil fatty acid methyltaurine sodium salt, N-myristoyl methyltaurine sodium salt, N-palmitoyl methyltaurine sodium salt, N-stearoyl methyltaurine sodium salt, N-stearoyl methyltaurine potassium salt, N-oleoyl methyltaurine sodium salt, N-cetyloyl methyltaurine potassium salt, and their non-neutralized forms.

12. A cosmetic raw material comprising a polyorganosiloxane micro-emulsion composition comprising (A) a polyorganosiloxane, (B) a material selected from the group consisting of (i) N-acylalkyltaurine and (ii) an N-acylalkyltaurine salt, and (C) water, and having an average particle size of emulsion particles less than 0.15 μm.

13. A cosmetic raw material comprising a polyorganosiloxane micro-emulsion composition comprising (A) a polyorganosiloxane, (B) a material selected from the group consisting of (i) N-acylalkyltaurine and (ii) an N-acylalkyltaurine salt, (C) water, and (D) a nonionic surface active agent, and having an average particle size of emulsion particles less than 0.15 μm.

14. A hair care cosmetic comprising in part, the material of claim 11.

15. A hair care cosmetic comprising in part, the material of claim 12.

16. A skin care cosmetic comprising in part, the material of claim 11.

17. A skin care cosmetic comprising in part, the material of claim 12.
